(19) United States
(12) Patent Application Publication
(10) Pub. No.: US 2003/0084520 A1
(43) Pub. Date: May 8, 2003

(54) Hair colouring compositions

(76) Inventors: Valerio Del Luca, Massa Lubrense (IT); Dean Ace Feebery, Staines Middlesex (GB); Carla Scorsini, Roma (IT); Catherine Margaret Strand, Berkshire (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/204,875

(22) PCT Filed: Jan. 17, 2001
(86) PCT No.: PCT/US01/01466

(30) Foreign Application Priority Data
Jan. 19, 2000 (GB) 0001233.6

(51) Int. Cl. A61K 7/13

(52) U.S. Cl. 8/405; 8/406; 8/407

(57) ABSTRACT

A hair colouring and/or bleaching composition comprising a peroxygen bleaching agent, an alkoxylated benzoic acid and an oxidative hair coloring agent. The hair colouring and/or bleaching compositions of the invention have excellent stability and provide improved overall hair colouring and/or bleaching performance.

### TECHNICAL FIELD

This invention relates to hair colouring and/or bleaching compositions, kits and methods for colouring hair and/or bleaching the hair, and more especially to hair colouring and/or bleaching compositions comprising a peroxygen oxidising agent, an alkoxylated benzoic acid and an oxidative hair coloring agent.

### BACKGROUND OF THE INVENTION

Over the years, various approaches to hair colouring and/or bleaching have been developed, these include, direct action dyes, natural dyes, metallic dyes and oxidative dyes.

Oxidative hair colouring agents can be used to deliver a variety of hair colours to the hair. To color human or animal hair using oxidative dye technology the hair is generally treated with a mixture of oxidative hair coloring agents and an oxidising agent. Generally the oxidising agent is present in a separate oxidising composition and the oxidising composition is mixed with a dye composition containing oxidative dyes to form a hair colouring and/or bleaching composition immediately before application of the latter to the hair. Hydrogen peroxide is the most commonly used oxidising agent. However, peroxygen oxidising agents have the inconvenience of being less stable compared to other types of oxidising agents.

Indeed, a major problem associated with compositions containing peroxygen oxidising agents, is their tendency to be unstable, especially upon storage, and more especially under conditions of elevated temperature. More particularly, it is believed that the peroxygen bleach (“oxidising agent”) present in such composition can decompose resulting in a reduction of the concentration of oxidising agent over time (“loss of available oxygen”). Loss of available oxygen in hair colouring compositions/kits consequently leads to the reduction of the overall hair colouring performance of the hair colouring and/or bleaching compositions. Loss of available oxygen can also lead to an undesirable volume increase in oxidising compositions and hair colouring compositions, particularly in products which have a high viscosity and where the oxygen cannot escape easily.

Thus there is a need for oxidative hair colouring and/or bleaching compositions which have stable peroxygen oxidising agents and which provide effective overall hair colouring and/or bleaching performance.

Oxidising compositions used in hair dyeing compositions and kits can be formulated comprising a radical scavenger in addition to the peroxygen oxidising agents. It is believed that radical scavengers act as a bleach stabilizer in such oxidising compositions. Examples of such compositions known in the art, include bleaching compositions used to treat fabrics and/or hard-surfaces and comprising a peroxygen bleach and a radical scavenger, as e.g., butyl hydroxy toluene (BHT) and the like (EP-A-0 791 362, EP-A-0 842 604, EP-A-0 842 606 and EP-A-0 843 001) or carpet treatment compositions comprising a peroxygen bleach and a radical scavenger, e.g., n-propyl gallate or butyl hydroxy toluene (BHT) and the like (EP-A-0 906 950).

However, the stability of the peroxygen oxidising agent in hair colouring and/or bleaching compositions and kits immediately after the manufacture of said hair colouring and/or bleaching composition containing a peroxygen oxidising agent or after manufacture of a separate oxidising composition comprising a peroxygen oxidising agent for use in a hair colouring and/or bleaching kit (“immediate chemical stability”) and the stability of the peroxygen bleach in said hair colouring compositions/kits upon prolonged periods of storage (“upon storage”) may still be further improved.

It is therefore an objective of the present invention to provide a peroxygen oxidising agent-containing hair dye composition, said composition being not only immediately chemically stable but also chemically stable upon storage, especially at higher temperatures. It is also an objective of the present invention to provide a hair colouring and/or bleaching kit comprising an oxidizing composition containing a peroxygen oxidising agent and an alkoxylated benzoic acid and a dye composition comprising an oxidative hair colouring agent wherein the oxidizing composition is not only immediately chemical stable but also chemically stable upon storage in the kit, especially at higher temperatures.

It has now been found that this objective can be met by a hair colouring and/or bleaching composition comprising an oxidative hair colouring agent, a peroxygen oxidising agent and an alkoxylated benzoic acid. It also also been found that this objective can be met by a hair colouring and/or bleaching kit comprising a first component which is an oxidising composition and a second component which is a dye composition wherein the oxidising composition comprises a peroxygen oxidising agent and an alkoxylated benzoic acid and wherein the dye composition comprises an oxidative hair colouring agent or mixture of oxidative hair colouring agents.

Advantageously, the hair colouring and/or bleaching compositions and kits as described herein also provide excellent overall hair colouring and/or bleaching performance. In addition, the hair colouring and/or bleaching compositions and kits of the present invention also minimize volume increases caused by loss of available oxygen, particularly in oxidising compositions and hair colouring and/or bleaching compositions having a thick rheology.

A further advantage of the oxidising compositions herein is that they are able to perform in a variety of conditions, i.e. when used neat or diluted.

All percentages are by weight of the final compositions in the form intended to be used unless specified otherwise, i.e. the final hair colouring and/or bleaching composition obtained by mixing an oxidising composition containing a peroxygen oxidising agent and an alkoxylated benzoic acid and a dye composition containing oxidative hair dyes.

### SUMMARY OF THE INVENTION

The subject of the present invention is a hair colouring and/or bleaching composition suitable for the treatment of human or animal hair.

According to one aspect of the present invention, there is provided a hair colouring and/or bleaching composition comprising an oxidative hair colouring agent, a peroxygen oxidising agent and an alkoxylated benzoic acid.

According to a further aspect of the present invention, there is provided a method for bleaching and/or colouring hair wherein the method comprises applying a hair bleaching and coloring composition applied directly to the hair, leaving the composition on the hair for 1 to 60 minutes, preferably 10 to 40 minutes, and then rinsing said composition from the hair and wherein said hair bleaching and/or colouring composition comprises an oxidative hair colouring agent, a peroxygen oxidising agent and an alkoxylated benzoic acid.

According to yet another aspect of the present invention, there is provided a kit comprising a first component and a second component, the first component being an oxidising composition and comprising a peroxygen bleaching agent and an alkoxylated benzoic acid, and the second component being a dye composition and comprising an oxidative hair colouring agent or mixture of oxidative hair colouring agents.

As used herein the term “oxidising composition” means a composition comprising a peroxygen oxidising agent and an alkoxylated benzoic acid and which is a separate composition from the dye composition, defined below.

As used herein the term “dye composition” means a composition comprising an oxidative hair colouring agent or a mixture of oxidative hair colouring agents and which is a separate composition from the oxidising composition, defined above.

As used herein the term “hair colouring and/or bleaching composition” means a composition comprising a peroxygen oxidising agent, an alkoxylated benzoic acid and on oxidative hair colouring agent. The hair colouring and/or bleaching composition can be obtained immediately before application to the hair by mixing of the oxidising composition and the dye composition, both defined above.

### DETAILED DESCRIPTION OF THE INVENTION

As used herein the term ‘hair’ to be treated may be ‘living’ i.e. on a living body or may be ‘non-living’ i.e. in a wig, hairpiece or other aggregation of non-living fibres, such as though used in textiles and fabrics. Mammalian, preferably human hair is preferred. However wool, fir and other melanin containing fibres are suitable substrates for the compositions according to the present invention.

As used herein the term ‘hair bleaching and coloring composition’ is used in the broad sense in that it is intended to encompass compositions containing the combinations herein of a peroxygen bleaching agent, an oxidative coloring agent and an alkoxylated benzoic acid. Moreover, it is also intended to include complex compositions which contain other components which may or may not be active ingredients. Thus, the term ‘hair bleaching and coloring composition’ is intended to apply to compositions which contain, in addition to a peroxygen bleaching agent, coloring agents and an alkoxylated benzoic acid, such things as, by way of example, oxidising aids, sequestrants, stabilisers, thickeners, buffers, carriers, surfactants, solvents, antioxidants, polymers, and conditioners.

Peroxygen Bleach Oxidising Agents

The compositions of the invention comprise as an essential feature at least one oxidising agent, which is a peroxygen bleach oxidising agent. The oxidising/bleaching agent is preferably present at a level of from about 0.01% to about 10%, preferably from about 0.01% to about 6%, more preferably from about 2% to about 5% by weight of final composition, i.e. the final hair colouring composition which is obtained by mixing an oxidising composition with a dye composition containing oxidative hair dyes.

Inorganic Oxidising Agents

A preferred oxidising agent for use herein is an inorganic peroxygen oxidising agent. The inorganic peroxygen oxidising agent should be safe and effective for use in the compositions herein. Preferably, the inorganic peroxygen oxidising agents suitable for use herein will be soluble in the compositions according to the present invention when in liquid form or in the form intended to be used. Preferably, inorganic peroxygen oxidising agents suitable for use herein will be water-soluble. Water soluble oxidising agents as defined herein means agents which have a solubility to the extent of about 10 g in 1000 ml of deionised water at 25° C. (“Chemistry” C. E. Mortimer. 5th Edn. p277).

The inorganic peroxygen oxidising agents useful herein are generally inorganic peroxygen materials capable of yielding peroxide in an aqueous solution, i.e. water-soluble sources of hydrogen peroxide. Inorganic peroxygen oxidising agents are well known in the art and include hydrogen peroxide, inorganic alkali metal peroxides such as sodium periodate, sodium perbromate and sodium peroxide, and inorganic perhydrate salt oxidising compounds, such as the alkali metal salts of perborates, percarbonates, perphosphates, persilicates, persulphates and the like. These inorganic perhydrate salts may be incorporated as monohydrates, tetrahydrates etc. Mixtures of two or more of such inorganic peroxygen oxidising agents can be used if desired. While alkali metal bromates and iodates are suitable for use herein the bromates are preferred. Highly preferred for use in the compositions according to the present invention is hydrogen peroxide.

Preformed Organic Peroxyacid

The compositions according to the present invention may instead or in addition to the inorganic peroxygen oxidising agent(s), comprise one or more preformed organic peroxyacid oxidising agents.

Suitable organic peroxyacid oxidising agents for use in the coloring compositions according to the present invention have the general formula:

R—C(O)OOH

wherein R is selected from saturated or unsaturated, substituted or unsubstituted, straight or branched chain, alkyl, aryl or alkaryl groups with from 1 to 14 carbon atoms.

A class of organic peroxyacid compounds suitable for use herein are the amide substituted compounds of the following general formulae:

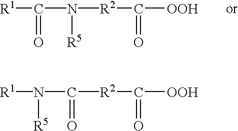

wherein R¹ is, a saturated or unsaturated alkyl or alkaryl group, or an aryl group, having from 1 to 14 carbon atoms, R² is, a saturated or unsaturated alkyl or alkaryl group, or an aryl group, having from 1 to 14 carbon atoms, and R⁵ is H or, a saturated or unsaturated alkyl or alkaryl group, or an aryl group, having from 1 to 10 carbon atoms. Amide substituted organic peroxyacid compounds of this type are described in EP-A-0,170,386.

Other suitable organic peroxyacid oxidising agents include persulphates such as monopersulpate, peracetic, pernanoic, nonylamidoperoxycaproic acid (NAPCA), perbenzoic, m-chloroperbenzoic, di-peroxy-isophthalic, mono-peroxyphthalic, peroxylauric, hexanesulphonyl peroxy propionic, N,N-pivaloylamino peroxycaproic, monoper succinic, nonanoyloxybenzoic, dodecanedioyl-monoperoxybenzoic, nonylamide of peroxyadipic acid, diacyl and tetraacylperoxides, especially diperoxydodecanedioic acid, diperoxytetradecanedioic acid and diperoxyhexadecanedioic acid and derivatives thereof Mono- and diperazelaic acid, mono- and diperbrassylic acid and N-phthaloylaminoperoxicaproic acid and derivatives thereof are also suitable for use herein.

The preformed organic peroxyacid oxidising agents should be safe and effective for use in the compositions herein. Preferably, the preformed organic peroxyacid oxidising agents suitable for use herein will be soluble in the compositions according to the present invention when in liquid form and in the form intended to be used. Preferably, organic peroxyacid oxidising agents suitable for use herein will be water-soluble. Water-soluble preformed organic peroxyacid oxidising agents as defined herein means agents which have a solubility to the extent of about 10 g in 1000 ml of deionised water at 25° C. (“Chemistry” C. E. Mortimer. 5th Edn. p277).

The preferred peroxyacid materials suitable for use herein are selected from peracetic and pernanoic acids and mixtures thereof.

When both an inorganic peroxygen oxidising agent and a preformed organic peroxy acid are present in the compositions herein, the weight ratio of the inorganic peroxygen oxidising agent to the preformed organic peroxy acid is preferably in the range of from about 0.0125:1 to about 500:1, more preferably from about 0.0125:1 to about 50:1.

In addition to the inorganic peroxygen oxidising agents and the preformed organic peroxyacid oxidising agents suitable for use herein, the compositions according to the present invention may optionally comprise additional organic peroxides such as urea peroxide, melamine peroxide and mixtures thereof.

Alkoxylated Benzoic Acid

As a second essential ingredient of the compositions according to the present invention comprise an alkoxylated benzoic acid or a salt thereof.

Generally, the alkoxylated benzoic acid or the salt thereof has the general formula:

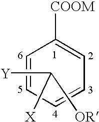

wherein: the substituents of the benzene ring X and Y are independently selected from —H, or —OR′; R′ is independently selected from C₁ to C₂₀ linear or branched alkyl chains, preferably R′ is independently selected from C₁ to C₅ linear or branched alkyl chains, more preferably R′ is —CH₃, and; M is hydrogen, a cation or a cationic moiety.

Preferably, M is selected from the group consisting of hydrogen, alkali metal ions and alkaline earth metal ions. More preferably, M is selected from the group consisting of hydrogen, sodium and potassium. Even more preferably, M is hydrogen.

In a preferred embodiment of the present invention, said alkoxylated benzoic acid or the salt thereof is a monoalkoxy benzoic acid or a salt thereof, wherein in the above general formula: the substituents of the benzene ring X and Y are —H; R′ is independently selected from C₁ to C₂₀ linear or branched alkyl chains, preferably R′ is independently selected from C₁ to C₅ linear or branched alkyl chains, more preferably R′ is —CH₃, and; M is hydrogen, a cation or a cationic moiety. Preferably, said monoalkoxy benzoic acid or a salt thereof is selected from the group consisting of o-/m-/p-methoxy benzoic acids, salts thereof, and mixtures thereof. More preferably, said monoalkoxy benzoic acid or a salt thereof is m-methoxy benzoic acid (wherein the methoxy group is in position 3 in the above general formula) or a salt thereof.

In another preferred embodiment of the present invention, said alkoxylated benzoic acid or the salt thereof is a dialkoxy benzoic acid or a salt thereof, wherein in the above general formula: the substituent of the benzene ring X is selected from —H; the substituent of the benzene ring Y is —OR′; R′ is independently selected from C₁ to C₂₀ linear or branched alkyl chains, preferably R′ is independently selected from C₁ to C₅ linear or branched alkyl chains, more preferably R′ is —CH₃, and; M is hydrogen, a cation or a cationic moiety.

In still another preferred embodiment of the present invention, said alkoxylated benzoic acid or the salt thereof is a trialkoxy benzoic acid or a salt thereof, wherein in the above general formula: the substituents of the benzene ring Y and X are —OR′; R′ is independently selected from C₁ to C₂₀ linear or branched alkyl chains, preferably R′ is independently selected from C₁ to C₅ linear or branched alkyl chains, more preferably R′ is —CH₃, and; M is hydrogen, a cation or a cationic moiety.

Preferably, said alkoxylated benzoic acid or a salt thereof, is selected from the group consisting of: a monoalkoxy benzoic acid, or a salt thereof, a dialkoxy benzoic acid, or a salt thereof; a trialkoxy benzoic acid, or a salt thereof, and a mixture thereof. More preferably, said alkoxylated benzoic acid or a salt thereof, is selected from the group consisting of: a dialkoxy benzoic acid, or a salt thereof; a trialkoxy benzoic acid, or a salt thereof; and a mixture thereof. Even more preferably, said alkoxylated benzoic acid or a salt thereof, is a trimethoxy benzoic acid or a salt thereof.

In a highly preferred embodiment of the present invention, said alkoxylated benzoic acid or the salt thereof is a trimethoxy benzoic acid or a salt thereof (TMBA), wherein in the above general formula: the substituents of the benzene ring Y and X are —OR′; R′ is —CH₃ and; M is hydrogen, a cation or a cationic moiety.

Preferably, said alkoxylated benzoic acid or the salt thereof is selected from the group consisting of 3,4,5,-trimethoxy benzoic acid, a salt thereof, 2,3,4-trimethoxy benzoic acid, a salt thereof, 2,4,5-trimethoxy benzoic acid, a salt thereof and a mixture thereof. More preferably, said alkoxylated benzoic acid or the salt thereof is 3,4,5,-trimethoxy benzoic acid or a salt thereof. Even more preferably, said alkoxylated benzoic acid or the salt thereof is 3,4,5,-trimethoxy benzoic acid.

Suitable monoalkoxy benzoic acids or salts thereof are commercially available from Aldrich, in particular m-methoxy benzoic acid is commercially available from Aldrich. Suitable trimethoxy benzoic acids or salts thereof are commercially available from Aldrich and Merck.

Typically, the bleaching composition according to the present invention may comprise from 0.001% to 5%, preferably from 0.005% to 2.5% and more preferably from 0.01% to 1.0% by weight of the total composition of said alkoxylated benzoic acid or a salt thereof.

It has now been found that an alkoxylated benzoic acid or a salt thereof, preferably a trialkoxy benzoic acid or a salt thereof, more preferably trimethoxy benzoic acid or a salt thereof (TMBA), acts as a radical scavenger in the oxidising composition or the hair colouring and/or bleaching composition according to the present invention, thereby not only stabilizing the peroxygen bleach in said compositions immediately after the manufacture of said compositions (“immediate chemical stability benefit”) but also stabilizing the peroxygen bleach in said compositions upon storage (“chemical stability upon storage benefit”). Indeed, the presence of an alkoxylated benzoic acid or a salt thereof, preferably a trialkoxy benzoic acid or a salt thereof, more preferably trimethoxy benzoic acid or a salt thereof (TMBA), in said compositions significantly increases the immediate chemical stability and particularly the chemical stability upon storage of the peroxygen bleach in said oxidising and hair colouring compositions. Although not wishing to be bound by theory, it is believed that decomposition of the peroxygen bleach (“parent peroxygen bleach”) and the interaction or reaction of the bleach with the other ingredients of the composition, as e.g., brighteners, surfactants, thickeners and the like, when present, proceeds via one electron chemistry. One electron chemistry reactions are initiated by the formation of free radicals from the parent peroxygen bleach. Generally, the radicals formed are more reactive and better oxidising species than the parent, accelerating the decomposition kinetics of the peroxygen bleach and its reaction or interaction with the other ingredients, when present in the composition. The result of such reactions is the decomposition of the peroxygen bleach and the other ingredients, when present. For example, where thickeners are components of the composition, decomposition thereof can be initiated by one electron processes, resulting in the loss or decrease of viscosity upon storage. To improve the immediate and/or storage stability of oxidising compositions and hair colouring and/or bleaching compositions and the compatibility of the peroxygen bleach with the other ingredients, when present, it is therefore important to reduce, slow down or preferably completely suppress one electron oxidation chemistry reactions. Alkoxylated benzoic acids or salts thereof, preferably trialkoxy benzoic acids or salts thereof, more preferably trimethoxy benzoic acid or a salt thereof (TMBA), have been found to exhibit an excellent performance in reduction, slowing down or complete suppression of said one electron oxidation chemistry reactions, particularly in peroxygen bleach-containing compositions.

Furthermore, it has been found that the “immediate chemical stability” and/or “the chemical stability upon storage” of the peroxygen bleach in the compositions herein and/or the other actives ingredients therein (where present), is improved when an alkoxylated benzoic acid or a salt thereof, preferably a trialkoxy benzoic acid or a salt thereof, more preferably trimethoxy benzoic acid or a salt thereof (TMBA), is present versus when it is not or when another radical scavenger, as e.g., butyl hydroxy toluene (BHT), n-propyl gallate and the like, is present.

By “immediate chemical stability” it is meant herein, that the peroxygen bleach in said oxidising compositions and said hair colouring and/or bleaching compositions is stable immediately after the manufacture of said compositions. By “chemical stability upon storage” it is meant herein, that the peroxygen bleach in oxidising compositions remains stable over a period of storage of 3 years at 25° C. Thus, in practice this means that an oxidising composition containing a peroxygen oxidising agent and an alkoxylated benzoic acid remains sufficiently stable throughout the shelf life of the hair colouring and/or bleaching product.

Generally, the immediate chemical stability and chemical stability upon storage of the peroxygen bleach in a bleaching composition may be evaluated by measuring the concentration of available oxygen (often abbreviated to AvO2) immediately after and at given storage times after having manufactured the composition. The % loss of available oxygen and/or the concentration of remaining available oxygen can be measured by chemical titration methods known in the art, such as the iodometric method, thiosulphatimetric method, the permanganometric method and the cerimetric method. Said methods and the criteria for the choice of the appropriate method are described for example in “Hydrogen Peroxide”, W. C. Schumb, C. N. Satterfield and R. L. Wentworth, Reinhold Publishing Corporation, New York, 1955 and “Organic Peroxides”, Daniel Swern, Editor Wiley Int. Science, 1970.

Hair Coloring Agents

The hair colouring and/or bleaching compositions of the present invention include as an essential feature an oxidative hair coloring agent. Such hair coloring agents are used in combination with the oxidising agent and an alkoxylated benzoic acid to formulate permanent, demi-permanent, semi-permanent or temporary hair dye compositions.

Permanent hair dye compositions as defined herein are compositions which once applied to the hair are substantially resistant to wash-out. Demi-permanent hair dye compositions as defined herein are compositions which are substantially removed from the hair after up to 24 washes. Semi-permanent hair dye compositions as defined herein are compositions which once applied to the hair are substantially removed from the hair after up to 10 washes. Temporary hair dye compositions as defined herein are compositions which once applied to the hair are substantially removed from the hair after up to 2 washes. These different types of hair coloring compositions can be formulated via the specific combination of oxidant and/or dyes at different levels and ratios. Wash out as defined herein is the process by which hair color is removed from the hair over time during normal hair cleansing regimen. Washfastness as defined herein, means, the resistance of the dyed hair to wash out.

Oxidative Hair Coloring Agents

The compositions herein comprise an oxidative hair coloring agent. The concentration of each oxidative hair coloring agent in the hair colouring and/or bleaching compositions according to the present invention is preferably from about 0.001% to about 3% by weight, more preferably from about 0.01% to about 2% by weight.

The total combined level of oxidative hair coloring agents in the hair colouring and/or bleaching compositions according to the present invention is from about 0.001% to about 5%, preferably from about 0.01% to about 4%, more preferably from about 0.1% to about 3%, most preferably from about 0.1% to about 1% by weight.

Any oxidative hair coloring agent can be used in the compositions according to the present invention. Typically, but without intending to be limited thereby, oxidative hair coloring agents, consist essentially of at least two components, which are collectively referred to as dye forming intermediates (or precursors). Dye forming intermediates can react in the presence of a suitable oxidant to form a colored molecule.

The dye forming intermediates used in oxidative hair colorants include: aromatic diamines, aminophenols, various heterocycles, phenols, napthols and their various derivatives. These dye forming intermediates can be broadly classified as; primary intermediates and secondary intermediates. Primary intermediates, which are also known as oxidative dye precursors, are chemical compounds which become activated upon oxidation and can then react with each other and/or with couplers to form colored dye complexes. The secondary intermediates, also known as color modifiers or couplers, are generally colorless molecules which can form colors in the presence of activated precursors/primary intermediates, and are used with other intermediates to generate specific color effects or to stabilise the color.

Primary intermediates suitable for use in the compositions and processes herein include: aromatic diamines, polyhydric phenols, amino phenols and derivatives of these aromatic compounds (e.g., N-substituted derivatives of the amines, and ethers of the phenols). Such primary intermediates are generally colorless molecules prior to oxidation.

While not wishing to be bound by any particular theory it is proposed herein that the process by which color is generated from these primary intermediates and secondary coupler compounds generally includes a stepwise sequence whereby the primary intermediate can become activated (by oxidation), and then enjoins with a coupler to give a dimeric, conjugated colored species, which in turn can enjoin with another ‘activated’ primary intermediate to produce a trimeric conjugated colored molecule.

Oxidative Dye Precursors

In general terms, oxidative dye primary intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear colored. For example, oxidative primary intermediates capable of forming colored polymers include materials such as aniline, which has a single functional group and which, on oxidation, forms a series of conjugated imines and quinoid dimers, trimers, etc. ranging in color from green to black. Compounds such as p-phenylenediamine, which has two functional groups, are capable of oxidative polymerization to yield higher molecular weight colored materials having extended conjugated electron systems. Oxidative dyes known in the art can be used in the compositions according to the present invention. A representative list of primary intermediates and secondary couplers suitable for use herein is found in Sagarin, “Cosmetic Science and Technology”, “Interscience, Special Ed. Vol. 2 pages 308 to 310. It is to be understood that the primary intermediates detailed below are only by way of example and are not intended to limit the compositions and processes herein.

The typical aromatic diamines, polyhydric phenols, amino phenols, and derivatives thereof, described above as primary intermediates can also have additional substituents on the aromatic ring, e.g. halogen, aldehyde, carboxylic acid, nitro, sulfonic acid and substituted and unsubstituted hydrocarbon groups, as well as additional substituents on the amino nitrogen and on the phenolic oxygen, e.g. substituted and unsubstituted alkyl and aryl groups.

Examples of suitable aromatic diamines, amino phenols, polyhydric phenols and derivatives thereof, respectively, are compounds having the general formulas (I), (II) and (III) below:

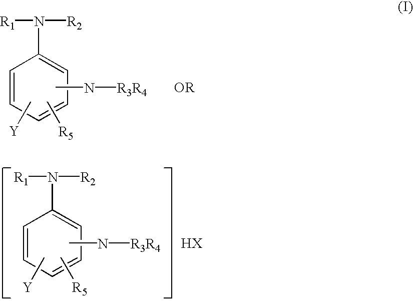

wherein Y is hydrogen, halogen, (e.g. fluorine, chlorine, bromine or iodine), nitro, amino, hydroxyl,

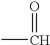

—COOM or —SO₃M (where M is hydrogen or an alkali or alkaline earth metal, ammonium, or substituted ammonium wherein one or more hydrogens on the ammonium ion is replaced with a 1 to 3 carbon atom alkyl or hydroxyalkyl radical), wherein R₁, R₂, R₃ and R₄ are the same or different from each other and are selected from the group consisting of hydrogen, C₁ to C₄ alkyl or alkenyl and C₆ to C₉ aryl, alkaryl or aralkyl, and R₅ is hydrogen, C₁ to C₄ unsubstituted or substituted alkyl or alkenyl wherein the substituents are selected from those designated as Y, above, or C₆ to C₉ unsubstituted or substituted aryl, alkaryl or aralkyl wherein the substituents are selected from those defined as Y, above. Since the precursors of formula (I) are amines, they can be used herein in the form of peroxide-compatible salts, as noted, wherein X represents peroxide-compatible anions of the type herein before detailed. The general formula of the salt indicated is to be understood to encompass those salts having mono-, di-, and tri-negative anions.

Specific examples of formula (I) compounds are: o-phenylenediamine, m-phenylenediamine, p-phenylenediamine, 2-chloro-p-phenylenediamine, 2-iodo-p-phenylenediamine, 4-nitro-o-phenylenediamine, 2-nitro-p-phenylenediamine, 1,3,5-triaminobenzene, 2-hydroxy-p-phenylenediamine, 2,4-diaminobenzoic acid, sodium 2,4-diaminobenzoate, calcium di-2,4-diaminobenzoate, ammonium 2,4-diaminobenzoate, trimethylammonium 2,4-, diaminobenzoate, tri-(2-hydroxyethyl)ammonium 2,4-diaminobenzoate, 2,4-diaminobenzaldehyde carbonate, 2,4-diaminobenzensulfonic acid, potassium 2,4-diaminobenzenesulfonate, N,N-diisopropyl-p-, phenylenediamine bicarbonate, N,N-dimethyl-p-phenylenediamine, N-ethyl-N′-(2-propenyl)-p-phenylenediamine, N-phenyl-p-phenylenediamine, N-phenyl-N-benzyl-p-phenylenediamine, N-ethyl-N′-(3-ethylphenyl)-p-phenylenediamine, 2,4-toluenediamine, 2-ethyl-p-phenylenediamine, 2-(2-bromoethyl)-p-phenylenediamine, 2-phenyl-p-phenylenediamine laurate, 4-(2,5-diaminophenyl)benzaldehyde, 2-benzyl-p-phenylenediamine acetate, 2-(4-nitrobenzyl)-p-phenylenediamine, 2-(4-methylphenyl)-p-phenylenediamine, 2-(2,5-diaminophenyl)-5-methylbenzoic acid, methoxyparaphenylenediamine, dimethyl-p-phenylenediamine, 2,5-dimethylpara-phenylenediamine, 2-methyl-5-methoxy-para-phenylenediamine, 2,6-methyl-5-methoxy-para-phenylenediamine, 3-methyl-4-amino-N,N-diethylaniline, N,N-bis(β-hydroxyethyl)-para-phenylenediamine, 3-methyl-4-amino-N,N-bis(β-hydroxyethyl)aniline, 3-chloro-4-amino-N,N-bis(β-hydroxyethyl)aniline, 4-amino-N-ethyl-N-(carbamethyl)aniline, 3-methyl-4-amino-N-ethyl-N-(carbamethyl)aniline, 4-amino-N-ethyl-(β-piperidonoethyl)aniline, 3-methyl-4-amino-N-ethyl-(β-piperidonoethyl)aniline, 4-amino-N-ethyl-N-(β-morpholinoethyl)aniline, 3-methyl-4-amino-N-ethyl-N-(β-morpholinoethyl)aniline, 4-amino-N-ethyl-N-(β-acetylaminoethyl)aniline, 4-amino-N-(β-methoxyethyl) aniline, 3-methyl-4-amino-N-ethyl-N-(β-acetylaminoethyl) aniline, 4-amino-N-ethyl-N-(β-mesylaminoethyl) aniline, 3-methyl-4-amino-N-ethyl-N-(β-mesylaminoethyl) aniline, 4-amino-N-ethyl-N-(β-sulphoethyl) aniline, 3-methyl-4-amino-N-ethyl-N-(β-sulphoethyl) aniline, N-(4-aminophenyl)morpholine, N-(4-aminophenyl)piperidine, 2,3-dimethyl-p-phenylenedianiine, isopropyl-p-phenylenediamine, N,N-bis-(2-hydroxyethyl)-p-phenylenediamine sulphate.

Compounds having the general structure (II) are as follows:

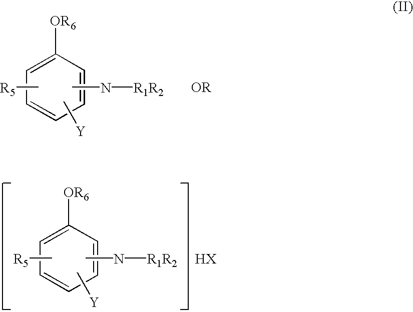

where X and Y are the same as in formula (I), R₁ and R₂ can be the same or different from each other and are the same as in formula (I), R₅ is the same as in formula (I) and R₆ is hydrogen or C₁ to C₄ substituted or unsubstituted alkyl or alkenyl wherein the substituents are selected from those defined as Y in formula (I).

Specific examples of formula (II) compounds are: o-aminophenol, m-aminophenol, p-aminophenol, 2-iodo-p-aminophenol, 2-nitro-p-aminophenol, 3,4-dihydroxyaniline, 3,4-diaminophenol, chloroacetate, 2-hydroxy-4-aminobenzoic acid, 2-hydroxy-4-aminobenzaldehyde, 3-amino-4-hydroxybenzenesulfonic acid, N,N-diisopropyl-p-aminophenol, N-methyl-N-(1-propenyl)-p-aminophenol, N-phenyl-N-benzyl-p-aminophenol sulphate, N-methyl-N-(3-ethylphenyl)-p-aminophenol, 2-nitro-5-ethyl-p-aminophenol, 2-nitro-5-(2-bromoethyl)-p-aminophenol, (2-hydroxy-5-aminophenyl)acetaldehyde, 2-methyl-p-aminophenol, (2-hydroxy-5-aminophenyl)acetic acid, 3-(2-hydroxy-5-aminophenyl)-1-propene, 3-(2-hydroxy-5-aminophenyl)-2-chloro-1-propene, 2-phenyl-p-aminophenol palmitate, 2-(4-nitrophenyl)-p-aminophenol, 2-benzyl-p-aminophenol, 2-(4-chlorobenzyl-p-aminophenol perchlorate, 2-(4-methylphenyl)-p-aminophenol, 2-(2-amino-4-methylphenyl)-p-aminophenol, p-methoxyaniline, 2-bromoethyl-4-aminophenyl ether phosphate, 2-nitroethyl-4-aminophenyl ether bromide, 2-aminoethyl-4-aminophenyl ether, 2-hydroxyethyl-4-aminophenyl ether, (4-aminophenoxy)acetaldehyde, (4-aminophenoxy)acetic acid, (4-aminophenoxy)methanesulfonic acid, 1-propenyl-4-aminophenyl ether isobutyrate, (2-chloro)-1-propenyl-4-aminophenyl ether, (2-nitro)-1-propenyl-4-aminophenyl ether, (2-amino)-propenyl-4-aminophenyl ether, (2-hydroxy)-1-propenyl-4-aminophenyl ether, N-methyl-p-aminophenol, 3-methyl-4-aminophenol, 2-chloro-4aminophenol, 3-chloro-4-aminophenol, 2,6-dimethyl-4-aminophenol, 3,5-dimethyl-4-aminophenol, 2,3-dimethyl-4-aminophenol, 2,5-dimethyl-4-aminophenol, 2-hydroxymethyl-4-aminophenol, 3-hydroxymethyl-4-aminophenol, 2,6-dichloro-4-aminophenol, 2,6-dibromo-4-aminophenol and 2-bromo-4-aminophenol.

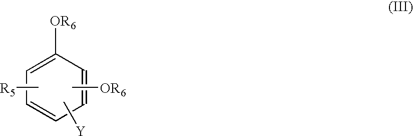

Specific examples of formula (III) compounds are: o-hydroxyphenol (catechol), m-hydroxyphenol (resorcinol), p-hydroxyphenol (hydroquinone), 4-methoxyphenol, 2-methoxyphenol, 4-(2-chloroethoxy) phenol, 4-(2-propenoxy) phenol, 4-(3-chloro-2-propenoxy) phenol, 2-chloro-4-hydroxyphenol (2-chlorohydroquinone), 2-nitro-4-hydroxyphenol(2-nitrohydroquinone), 2-amino-4-hydroxyphenol, 1,2,3-trihydroxybenzene (pyrogallol), 2,4-dihydroxybenzaldehyde, 3,4-dihydoxybenzoic acid, 2,4-dihydroxybenzenesulfonic acid, 3-ethyl-4hydroxyphenol, 3-(2-nitroethyl)-4-hydroxyphenol, 3-(2-propenyl)-4-hydroxyphenol, 3-(3-chloro-2-propenyl)-4-hydroxyphenol, 2-phenyl-4-hydroxyphenol, 2-(4-chlorophenyl)-4-hydroxyphenol, 2-benzyl-4-hydroxyphenol, 2-(2-nitrophenyl)-4-hydroxyphenol, 2-(2-methylphenyl)-4-hydroxyphenol, 2-(2-methyl-4-chlorophenyl)-4-hydroxyphenol, 3-methoxy-4-hydroxy-benzaldehyde, 2-methoxy-4-(1-propenyl)phenol, 4-hydroxy-3-methoxycinnamic acid, 2,5-dimethoxyaniline, 2-methylresorcinol, alpha napthol and salts thereof.

Secondary coupling compounds which are suitable for inclusion in the coloring compositions and processes herein before described include certain aromatic amines and phenols and derivatives thereof which do not produce color singly, but which modify the color, shade or intensity of the colors developed by the primary oxidized dye intermediates. Certain aromatic amines and phenolic compounds, and derivatives thereof, including some aromatic diamines and polyhydric phenols of the types described by formulas (I), (Ia), (Ib), (II) and (III) above, but which are well known in the art not to be suitable primary intermediates, are suitable as couplers herein. Polyhydric alcohols are also suitable for use as couplers herein.

The aromatic amines and phenols and derivatives described above as couplers can also have additional substituents on the aromatic ring, e.g., halogen, aldehyde, carboxylic acid, nitro, sulfonyl and substituted and unsubstituted by hydrocarbon groups, as well as additional substituents on the amino nitrogen, or phenolic oxygen, e.g. substituted and unsubstituted alkyl and aryl groups. Again, peroxide-compatible salts thereof are suitable for use herein.

Examples of aromatic amines, phenols and derivatives thereof are compounds of the general formulas (IV) and (V) below:

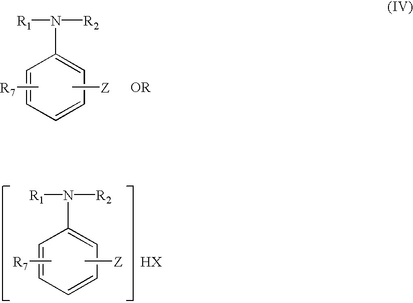

wherein Z is hydrogen, C₁ and C₃ alkyl, halogen (e.g. fluorine, chlorine, bromine or iodine) nitro,

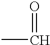

—COOM or SO₃M, (where M is hydrogen or an alkali or alkaline earth metal, ammonium or substituted ammonium wherein one or more hydrogens on the ammonium ion is replaced with a 1 to 3 carbon atom alkyl or hydroxyalkyl radical), wherein R₁ and R₂ are the same or different and are selected from the group consisting of hydrogen, C₁ to C₄ alkyl or alkenyl and C₆ to C₉ aryl, alkaryl or aralkyl and R₇ is hydrogen, C₁ to C₄ unsubstituted or substituted alkyl or alkenyl wherein the substituents are selected from those designated as Z above or C₆ to C₉ unsubstituted or substituted aryl, alkaryl or aralkyl wherein the substituents are selected from those defined as Z above and wherein X is as defined in formula (I).

Specific examples of formula (IV) compounds are: aniline, p-chloroaniline, p-fluoroaniline, p-nitroaniline, p-aminobenzaldehyde, p-aminobenzoic acid, sodium-p-aminobenzoate, lithium-p-aminobenzoate, calcium di-p-aminobenzoate, ammonium-p-aminobenzoate, trimethylammonium-p-aminobenzoate, tri(2-hydroxyethyl)-p-aminobenzoate, p-aminobenzenesulfonic acid, potassium p-aminobenzenesulfonate, N-methylaniline, N-propyl-N-phenylaniline, N-methyl-N-2-propenylaniline, N-benzylaniline, N-(2-ethylphenyl)aniline, 4-methylaniline, 4-(2-bromoethyl)aniline, 2-(2-nitroethyl)aniline, (4-aminophenyl)acetaldehyde, (4-aminophenyl)acetic acid, 4-(2-propenyl)aniline acetate, 4-(3-bromo-2-propenyl)aniline, 4-phenylaniline chloroacetate, 4-(3-chlorophenyl)aniline, 4-benzylaniline, 4-(4-iodobenzyl)aniline, 4-(3-ethylphenyl)aniline, 4-(2-chloro-4-ethylphenyl)aniline.

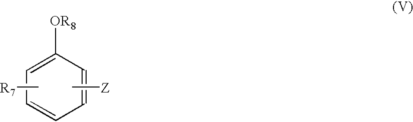

wherein Z and R₇ are defined as in formula (IV) and R₈ is hydrogen or C₁ to C₄ substituted or unsubstituted alkyl or alkenyl wherein the substituents are selected from those defined as Z in formula (IV).

Specific examples of formula (V) compounds are: phenol, p-chlorophenol, p-nitrophenol, p-hydroxybenzaldehyde, p-hydroxybenzoic acid, p-hydroxybenzenesulfonic acid, ethylphenyl ether, 2-chloroethylphenyl ether, 2-nitroethylphenyl ether, phenoxyacetaldehyde, phenoxyacetic acid, 3-phenoxy-1-propene, 3-phenoxy-2-nitro-1-propene, 3-phenoxy-2-bromo-1-propene, 4-propylphenol, 4-(3-bromopropyl)phenol, 2-(2-nitroethyl)phenol, (4-hydroxyphenyl)acetaldehyde, (4-hydroxyphenyl)acetic acid, 4-(2-propenyl)phenol, 4-phenylphenol, 4-benzylphenol, 4-(3-fluoro-2-propenyl)phenol, 4-(4-chlorobenzyl)phenol, 4-(3-ethylphenyl)phenol, 4-(2-chloro-3-ethylphenyl)phenol, 2,5-xylenol, 2,5-diaminopyridine, 2-hydroxy-5-aminopyridine, 2-amino-3-hydroxy pyridine, tetraaminopyrimindine, 1,2,4-trihydroxybenzene, 1,2,4-trihydroxy-5-(C₁-C₆-alkyl)benzene, 1,2,3-trihydroxybenzene, 4-aminoresorcinol, 1,2-dihydroxybenzene, 2-amino-1,4-dihydroxybenzene, 2-amino-4-methoxy-phenol, 2,4-diaminophenol, 3-methoxy-1,2-dihydroxy-benzene, 1,4-dihydroxy-2-(N,N-diethylamino)benzene, 2,5-diamino-4-methoxy-1-hydroxybenzene, 4,6-dimethoxy-3-amino-1-hydroxybenzene, 2,6-dimethyl-4-[N-(p-hydroxyphenyl)amino]-1-hydroxybenzene, 1,5-diamino-2-methyl-4-[N-(p-hydroxyphenyl)amino]benzene and salts thereof.

Additional primary intermediates suitable for use herein include catechol species and in particular catechol “dopa” species which includes dopa itself as well as homologs, analogs and derivatives of DOPA. Examples of suitable cachetol species include cysteinyl dopa, alpha alkyl dopa having 1 to 4 , preferably 1 to 2 carbon atoms in the alkyl group, epinephrine and dopa alkyl esters having 1 to 6 , preferably 1 to 2 carbon atoms in the alkyl group.

In general suitable catechols are represented by formula (VI) below:

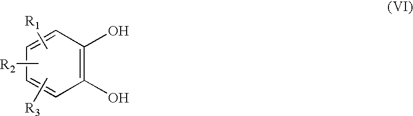

wherein R₁, R₂ and R₃, which may be the same or different, are electron donor or acceptor substutuents selected from H, lower (C₁-C₆) alkyl, OH, OR, COOR, NHCOR, CN, COOH, Halogen, NO₂, CF₃, SO₃H or NR₄R₅, with the proviso that only one of the R₁, R₂ or R₃ can be CN, COOH, halogen, NO₂, CF₃ or SO₃H: R₄ and R₅, which may be the same or different, are H, lower (C₁-C₆) alky or substituted lower (C₁-C₆) alkyl in which the substituent may be OH, OR, NHCOR₆, NHCONH₂, NHCO₂R₆, NHCSNH₂, CN, COOH, SO₃H, SO₂NR₆, SO₂R₆ or CO₂R₆; R₆ is lower (C₁-C₆) alkyl, lower (C₁-C₆) hydroxyalkyl phenyl linked to the nitrogen by an aliylene chain, phenyl or substituted phenyl with the substituent defined as R₁, and R is C₁-C₆ alkyl or C₁-C₆ hydroxyalkyl.

Also included herein are oxidative hair coloring agents of the formula:

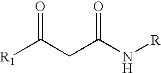

wherein: R₁=substituted or unsubstituted benzene ring, tertiary-butyl, etc.; R=substituted or unsubstituted benzene ring and the formula:

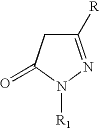

wherein R=aminoalkyl, amidoalkyl, aminobenzene (substituted or unsubstituted), amidobenzene (substituted or unsubstituted), alkyl, substituted or unsubstituted benzene ring; R₁=substituted or unsubstituted benzene ring.

The primary intermediates can be used herein alone or in combination with other primary intermediates, and one or more can be used in combination with one or more couplers. The choice of primary intermediates and couplers will be determined by the color, shade and intensity of coloration which is desired. There are nineteen preferred primary intermediates and couplers which can be used herein, singly or in combination, to provide dyes having a variety of shades ranging from ash blonde to black; these are: pyrogallol, resorcinol, p-toluenediamine, p-phenylenediamine, o-phenylenediarnine, m-phenylenediamine, o-aminophenol, p-aminophenol, 4-amino-2-nitrophenol, nitro-p-phenylenediamine, N-phenyl-p-phenylenediamine, m-aminophenol, 2-amino-3-hydroxypyridine, 1-napthol, N,N bis (2-hydroxyethyl)p-phenylenediamine, 4-amino-2-hydroxytoluene, 1,5-dihydroxynapthalene, 2-methyl resorcinol and 2,4-diaminoanisole. These can be used in the molecular form or in the form of peroxide-compatible salts, as detailed above.

For example low intensity colors such as natural blond to light brown hair shades generally comprise from about 0.001% to about 5%, preferably from about 0.1% to about 2%, more preferably from about 0.2% to about 1% by weight of coloring composition of total oxidative dyeing agents and may be achieved by the combination of primary intermediates such as 1,4-diamino-benzene, 2,5-diamino toluene, 2,5-diamino-anisole, 4-aminophenol, 2,5-diamino-benzyl alcohol and 2-(2′,5′-diamino)phenyl-ethanol with couplers such as resorcinol, 2-methyl resorcinl or 4-chloro resorcinol.

Similarly combination of the above primary intermediates with couplers, such as, 5-amino-2-methyl phenol and 1,3-diamino-benzene derivatives such as 2,4-diamino-anisole at levels of from about 0.5% to about 1% of total dyeing agents can lead to medium intensity red colors. High intensity colors such as blue to blue-violet hair shades can be produced by the combination of the above primary intermediates with couplers such as 1,3-diamino-benzene or its derivatives such as 2,5-diamino-toluene at levels of from about 1% to about 6% by weight of composition of total dyeing agents. Black hair colors can be obtained by combining the aforementioned primary intermediates with couplers such as 1,3-diaminobenzene or its derivatives

Non-oxidative and Other Dyes

The hair coloring compositions of the present invention may, in addition to an oxidative hair coloring agent, include non-oxidative and other dye materials, such as those disclosed in WO98/27945. Optional non-oxidative and other dyes suitable for use in the hair coloring compositions and processes according to the present invention include both semi-permanent, temporary and other dyes. Non-oxidative dyes as defined herein include the so-called ‘direct action dyes’, metallic dyes, metal chelate dyes, fibre reactive dyes and other synthetic and natural dyes. Various types of non-oxidative dyes are detailed in: ‘Chemical and Physical Behaviour of Human Hair’ 3rd Ed. by Clarence Robbins (pp250-259); ‘The Chemistry and Manufacture of Cosmetics’. Volume IV. 2nd Ed. Maison G. De Navarre at chapter 45 by G. S. Kass (pp841-920); ‘cosmetics: Science and Technology’ 2nd Ed., Vol. II Balsam Sagarin, Chapter 23 by F. E. Wall (pp 279-343); ‘The Science of Hair Care’ edited by C. Zviak, Chapter 7 (pp 235-261) and ‘Hair Dyes’, J. C. Johnson, Noyes Data Corp., Park Ridge, U.S.A. (1973), (pp 3-91 and 113-139).

pH

The hair colouring and/or bleaching compositions of the present invention can be formulated over a wide pH range, for example, 2-12. The hair colouring and/or bleaching compositions herein can be formulated at low pH, preferably having a pH in the range of from about 2 to about 7 , preferably from about 3 to about 6, more preferably 3 to 5.8, even more preferably 3.5 to 5.5, and especially 3.5 to 5, and also as high pH compositions having a pH in the range of from about 8 to about 10.

If so desired, and in order to adjust the pH of the composition, the compositions may contain one or more optional buffering agents and/or hair swelling agents (HSAs). Several different pH modifiers can be used to adjust the pH of the final composition or any constituent part thereof. However, preferred compositions herein are substantially free of additional buffering agents, buffering agents and hair swelling agents, i.e. they comprise less than about 1%, preferably less than about 0.5%, more preferably less than about 0.1% by weight of such agents.

This pH adjustment can be effected by using well known acidifying agents in the field of treating keratinous fibres, and in particular human hair, such as inorganic and organic acids such as hydrochloric acid, tartaric acid, citric acid, succinic acid, phosphoric acid and carboxylic or sulphonic acids such as ascorbic acid, acetic acid, lactic acid, sulphuric acid, formic acid, ammonium sulphate and sodium dihydrogenphosphate/phosphoric acid, disodium hydrogenphosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid, succinic acid and mixtures thereof.

Examples of alkaline buffering agents are ammonium hydroxide, ethylamine, dipropylamine, triethylamine and alkanediamines such as 1,3-diaminopropane, anhydrous alkaline alkanolamines such as, mono or di- ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-argenine, lysine, alanine, leucine, iso-leucine, oxylysine and histidine and alkanolamines such as dimethylaminoethanol and aminoalkylpropanediol and mixtures thereof. Also suitable for use herein are compounds other than ammonium carbonate or ammonium carbamate that form HCO₃— by dissociation in water (hereinafter referred to as ‘ion forming compounds’). Examples of suitable ion forming compounds are Na₂CO₃, NaHCO₃, K₂CO₃, (NH₄)₂CO₃ NH₄HCO₃, CaCO₃ and Ca(HCO₃) and mixtures thereof.

The hair colouring and/or bleaching compositions according to the present invention, may, as will be described later herein, be comprised of a final solution containing oxidising agent, hair coloring agent and an alkoxylated benzoic acid which have been admixed prior to application to the hair or a single component system. As such, the compositions according to the present invention may comprise coloring kits of a number of separate components.

In oxidising and coloring kits comprising a portion of inorganic peroxygen oxidising agent, such as hydrogen peroxide, which may be present in either solid or liquid form, a buffering agent solution can be used to maintain a desired pH. Since hydrogen peroxide is most stable in the pH range from 2 to 4, it is preferable to use a buffering agent having a pH within this range. Dilute acids are suitable hydrogen peroxide buffering agents.

Thickeners

The hair colouring and/or bleaching compositions of the present invention may additionally include a thickener at a level of from about 0.05% to about 20%, preferably from about 0.1% to about 10%, more preferably from about 0.5% to about 5% by weight. Thickening agents suitable for use in the compositions herein are selected from oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM), steareth-20 methacrylate copolymer; Aculyn 44 (RTM), polyurethane resin and Acusol 830 (RTM), acrylates copolymer which are available from Rohm and Haas, Philadelphia, Pa., USA. Additional thickening agents suitable for use herein include sodium alginate or gum arabic, or cellulose derivatives, such as methyl cellulose or the sodium salt of carboxymethylcellulose or acrylic polymers.

Heavy Metal Ion Sequestrant

The coloring compositions of the invention may contain as an optional but preferred component a heavy metal ion sequestrant. By heavy metal ion sequestrant it is meant herein components which act to sequester (chelate or scavenge) heavy metal ions. These components may also have calcium and magnesium chelation capacity, but preferentially they show selectivity to binding heavy metal ions such as iron, manganese and copper. Such sequestering agents are valuable in hair coloring compositions herein in combination with the alkoxylated benzoic acid for the delivery of controlled oxidising action as well as for the provision of good storage stability of the hair coloring products.

Heavy metal ion sequestrants are generally present at a level of from about 0.005% to about 20%, preferably from about 0.01% to about 10%, more preferably from about 0.05% to about 2% by weight of the compositions.

Various sequestering agents, including the amino phosphonates, available as Dequest (RTM) from Monsanto, the nitriloacetates, the hydroxyethyl-ethylene triamines and the like are known for such use. Suitable heavy metal ion sequestrants for use herein include organic phosphonates, such as the amino alkylene poly (alkylene phosphonates), alkali metal ethane 1-hydroxy disphosphonates and nitrilo trimethylene phosphonates.

Preferred among the above species are diethylene triamine penta (methylene phosphonate), ethylene diamine tri (methylene phosphonate) hexamethylene diamine tetra (methylene phosphonate) and hydroxy-ethylene 1,1 diphosphonate.

Preferred biodegradable non-phosphorous heavy metal ion sequestrants suitable for use herein include nitrilotriacetic acid and polyaminocarboxylic acids such as ethylenediaminotetracetic acid, ethylenetriamine pentaacetic acid, ethylenediamine disuccinic acid, ethylenediamine diglutaric acid, 2-hydroxypropylenediamine disuccinic acid or any salts thereof. Especially preferred is ethylenediamine-N,N′-disuccinic acid (EDDS). see U.S. Pat. No. 4,704,233, or the alkali metal, alkaline earth metal, ammonium, or substituted ammonium salts thereof, or mixtures thereof.

Other suitable heavy metal ion sequestrants for use herein are iminodiacetic acid derivatives such as 2-hydroxyethyl diacetic acid or glyceryl imino diacetic acid, described in EP-A-317,542 and EP-A-399,133. The iminodiacetic acid-N-2-hydroxypropyl sulfonic acid and aspartic acid N-carboxymethyl N-2-hydroxypropyl-3-sulfonic acid sequestrants described in EP-A-516,102 are also suitable herein. The β-alanine-N,N′-diacetic acid, aspartic acid-N,N′-diacetic acid, aspartic acid-N-monoacetic acid and iminodisuccinic acid sequestrants described in EP-A-509,382 are also suitable.

EP-A-476,257 describes suitable amino based sequestrants. EP-A-510,331 describes suitable sequestrants derived from collagen, keratin or casein. EP-A-528,859 describes a suitable alkyl iminodiacetic acid sequestrant. Dipicolinic acid and 2-phosphonobutane-1,2,4-tricarboxylic acid are also suitable. Glycinamide-N,N′-disuccinic acid (GADS), ethylenediamine-N-N′-diglutaric acid (EDDG) and 2-hydroxypropylenediamine-N-N′-disuccinic acid (HPDDS) are also suitable.

The heavy metal ion sequestering agents of the present invention may be used in their alkali or alkaline earth metal salts.

Particularly preferred combinations of heavy metal ion sequestering agents for use herein include (a) sodium stannate, etidronic acid, and pentasodium pentatate, (b) sodium stannate, disodium pyrophosphate, sodium pentatate, © sodium stannate, disodium pyrophosphate and EDTA, (d) sodium stannate, etidronic acid and EDTA and (e) sodium stannate, disodium pyrophosphate and etidronic acid.

Diluent

Water is the preferred diluent for the compositions according to the present invention. However, the compositions according to the present invention may include one or more solvents as additional diluent materials. Generally, solvents suitable for use in the coloring compositions of the present invention are selected to be miscible with water and innocuous to the skin. Solvents suitable for use as additional diluents herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a preferred group includes ethanol, isopropanol, n-propanol, butanol, propylene glycol, ethylene glycol monoethyl ether, and mixtures thereof. Water is the preferred principal diluent in the compositions according to the present invention. Principal diluent, as defined herein, means, that the level of water present is higher than the total level of any other diluents.

The diluent is present at a level preferably of from about 5% to about 99.98%, preferably from about 15% to about 99.5%, more preferably at least from about 30% to about 99%, and especially from about 50% to about 98% by weight of the compositions herein.

Surfactant Materials

The compositions of the present invention can additionally contain a surfactant system. Suitable surfactants for inclusion in the compositions of the invention generally have a lipophilic chain length of from about 8 to about 22 carbon atoms and can be selected from anionic, cationic, nonionic, amphoteric, zwitterionic surfactants and mixtures thereof. Suitable surfactants for use herein include those disclosed in WO98/27945.

Optional Materials

A number of additional optional materials can be added to the coloring compositions herein described each at a level of from about 0.001% to about 5%, preferably from about 0.01% to about 3%, more preferably from about 0.05% to about 2% by weight of composition. Such materials include proteins and polypeptides and derivatives thereof; water-soluble or solubilizable preservatives such as DMDM Hydantoin, Germall 115, methyl, ethyl, propyl and butyl esters of hydroxybenzoic acid, EDTA, Euxyl (RTM) K400, natural preservatives such as benzyl alcohol, potassium sorbate and bisabalol, benzoic acid, sodium benzoate and 2-phenoxyethanol; antioxidants such as sodium sulphite, hydroquinone, sodium bisulphite, sodium metabisulphite and thyoglycolic acid, sodium dithionite, erythrobic acid and other mercaptans; dye removers such as oxalic acid, sulphated castor oil, salicylic acid and sodium thiosulphate; H₂O₂ stabilisers such as tin compounds such as sodium stannate, stannic hydroxide and stannous octoate, acetanilide, phenacetin colloidal silica such as magnesium silicate, oxyquinoline sulphate, sodium phosphate, and tetrasodium pyrophosphate; and ρ-hydroxybenzoates; moisturising agents such as hyaluronic acid, chitin , and starch-grafted sodium polyacrylates such as Sanwet (RTM) IM-1000, IM-1500 and IM-2500 available from Celanese Superabsorbent Materials, Portsmith, Va., USA and described in U.S. Pat. No. 4,076,663 as well as methyl cellulose, starch, higher fatty alcohols, paraffin oils, fatty acids and the like; solvents; anti-bacterial agents such as Oxeco (phenoxy isopropanol); low temperature phase modifiers such as ammonium ion sources (e.g. NH₄ Cl); viscosity control agents such as magnesium sulfate and other electrolytes; quaternary amine compounds such as distearyl-, dilauryl-, di-hydrogenated beef tallow-, dimethyl ammonium chloride, dicetyldiethyl ammoniumethylsulphate, ditallowdimethyl ammonium methylsulphate, disoya dimethyl ammonium chloride and dicoco dimethyl ammonium chloride; hair conditioning agents such as silicones, higher alcohols, cationic polymers and the like; enzyme stabilisers such as water soluble sources of calcium or borate species; colouring agents; TiO₂ and TiO₂-coated mica; perfumes and perfume solubilizers; and zeolites such as Valfour BV400 and derivatives thereof and Ca²⁺/Mg²⁺ sequestrants such as polycarboxylates, amino polycarboxylates, polyphosphonates, amino polyphosphonates etc., metal catalyts, such as transition metal catalysts, heavy metal ion sequestrants, enzymes, and water softening agents such as sodium citrate.

Packaging

The compositions of the present invention can be packaged in any package which is suitable for use by a consumer. In particular, the packaging used can be such as to accommodate a single hair colouring and/or bleaching composition or can be such as to accommodate a separate oxidising composition and a separate dye composition which are intended to be stored separately and mixed immediately prior to application to the hair. The packaging can be for example in the form of an aerosol or a “bag-in-bottle” having one chamber to accommodate an oxidising composition and a second chamber to accommodate a dye composition and means for mixing the two separate compositions prior to application on the hair. A preferred package for use with the compositions herein is a “bag-in-bottle” package such as those described in WO99/26508, WO99/26509, WO99/26510, WO99/26511 and WO99/26596.

The present invention is represented by the following non-limiting examples. In the examples, all concentrations are on a 100% active basis and all percentages are by weight unless otherwise stated and the abbreviations have the following designations:

### EXAMPLES I-VI

The following Examples I-VI are oxidising compositions comprising hydrogen peroxide and 3,4,5-trimethoxy benzoic acid. The percentages given in Examples I-VI are by weight of the oxidising composition.

| Ingredient | I/% | II/% | III/% | IV/% | V/% | VI/% |
|---|---|---|---|---|---|---|
| Pentasodium Pentatate | 0.1 | 0.1 | 0.1 | 0.1 | 0.1 | 0.1 |
| Sodium Stannate | 0.04 | 0.04 | 0 | 0 | 0.04 | 0.04 |
| 3,4,5-trimethoxybenzoic | 0.05 | 0.05 | 0.05 | 0.05 | 0.05 | 0.05 |
| acid | | | | | | |
| Etidronic acid | 0.1 | 0.1 | 0.1 | 0.1 | 0 | 0 |
| Disodium Pyrophosphate | 0 | 0 | 0 | 0 | 0.1 | 0.1 |
| Hydrogen Peroxide | 6 | 9 | 6 | 9 | 6 | 9 |
| Sodium Benzoate | 0.09 | 0.09 | 0.09 | 0.09 | 0.09 | 0.09 |
| Phenoxyethanol | 0.11 | 0.11 | 0.11 | 0.11 | 0.11 | 0.11 |
| Tetrasodium EDTA | 0.03 | 0.03 | 0.03 | 0.03 | 0.03 | 0.03 |
| Ceteareth-25 | 1.5 | 1.5 | 1.5 | 1.5 | 1.5 | 1.5 |
| Cetyl Alcohol | 2.25 | 2.25 | 2.25 | 2.25 | 2.25 | 2.25 |
| Stearyl Alcohol | 2.25 | 2.25 | 2.25 | 2.25 | 2.25 | 2.25 |
| Citric Acid | 0.5 | 0.5 | 0.15 | 0.15 | 0.36 | 0.36 |
| Sodium Hydroxide | 0.09 | 0.09 | 0.09 | 0.09 | 0.09 | 0.09 |

| | |
|---|---|
| Water and minors | to 100 |

Examples I-VI provide oxidising compositions having excellent stability and which minimise undesirable volume increases.

### EXAMPLES VII-XI

The following Examples VII-XI are oxidative compositions comprising oxidative hair dyes. The percentages given in Examples VII-XI are by weight of the oxidative dye compositions.

| Ingredient | VI/I% | VIII/% | IX/% | X/% | XI/% |
|---|---|---|---|---|---|
| Cetyl Alcohol | 2.25 | 2.25 | 2.25 | 2.25 | 2.25 |
| Stearyl Alcohol | 2.25 | 2.25 | 2.25 | 2.25 | 2.25 |
| Ceteareth-25 | 1.5 | 1.5 | 1.5 | 1.5 | 1.5 |
| Paraphenylene diamine | 0.8 | 0.34 | 0.2 | 2 | 0 |
| para-aminophenol | 0.6 | 0.34 | 1.5 | 0 | 0 |
| 2-amino-3-hydroxy | 0.6 | 0 | 0.6 | 0 | 0 |
| pyridine | | | | | |
| 1-naphthol | 0 | 0 | 0 | 0 | 0 |
| resorcinol | 0 | 0 | 0 | 0.5 | 0 |
| 4-amino-2-hydroxy | 0 | 0 | 0 | 0 | 0.2 |
| toluene | | | | | |
| meta-phenylene diamine | 0 | 0 | 0 | 0.4 | 0 |
| meta-aminophenol | 0 | 0 | 0 | 0 | 0 |
| ortho-aminophenol | 0 | 0.34 | 0 | 0 | 0.2 |
| sodium sulphite | 0.2 | 0.2 | 0.2 | 0.2 | 0.2 |
| ascorbic acid | 0.6 | 0 | 0.6 | 1.6 | 0 |
| tetrasodium edta | 0.24 | 0.24 | 0.24 | 0.24 | 0.24 |
| sodium benzoate | 0.09 | 0.09 | 0.09 | 0.09 | 0.09 |
| phenoxyethanol | 0.11 | 0.11 | 0.11 | 0.11 | 0.11 |
| citric acid | 1 | 1.6 | 1.6 | 1.6 | 1.6 |
| sodium hydroxide | to pH 5 | to pH 5 | to pH 5 | to pH 5 | to pH 5 |

| | |
|---|---|
| water and minors | to 100 |

The Examples above can be used in various ways to colour the hair. In a preferred embodiment an oxidising composition (any of Example I-VI) is mixed with on oxidative dye composition (any of Examples VII-XI) in a 1:1 ratio to obtain a hair colouring and/or bleaching composition which is then applied to the hair.

In the examples above, water is used as the diluent. However in variations hereof water can be replaced, in part, by from about 0.5% to about 50% by weight of the total water content of the examples by diluents such as lower alcohols, e.g., ethylene glycol, ethylene glycol monoethyl ether, diethylene glycol, diethylene glycol monoethyl ether, propylene glycol, 1,3-propanediol, ethanol, isopropyl alcohol, glycerine, butoxyethanol, ethoxydiglycol, hexylene glycol, polyglyceryl-2-oleyl ether and mixtures thereof.

Examples I-XI (when mixed together as described above to obtain a hair colouring and/or bleaching composition) provide a complete range of colours, i.e. from blondes to blacks and have excellent product stability, while reducing skin irritation, hair damage and odor. The hair colouring and/or bleaching compositions of the examples above also provide excellent grey coverage.

Method of Use

It is important that dyeing compositions be in a form which is easy and convenient to prepare and use by the consumer, since the oxidising agent must remain in contact with the hair for a certain period of time and not run or drip off of the hair, possibly causing eye or skin irritation.

To address the above, the coloring compositions of the present invention can be provided in both a single pack or in kit form as separately packaged components to maintain stability, and, if so desired, either mixed by the user immediately prior to application to the hair, or mixed and stored for future use, or mixed and partly used and the remainder stored for future use. Separately packaged components can be delivered from a single package having multi-compartments to keep each component separate until it is mixed. Suitable single packages having multiple components include those disclosed in WO99/26508, WO99/26509, WO99/26510, WO99/26511 and WO99/26596.

As hereinbefore described, the compositions according to the present invention may be used by the consumer as a single component package. Such a single pack would comprise a single solution containing the oxidising agent, the hair coloring agent and the alkoxylated benzoic acid. The solution would be applied directly to the hair by the consumer without the need for any pretreatments or mixing thereby providing a simple, fast, easy to use, ‘no-mess’ hair coloring system. A further advantage of such a single component system is that it could be stored and re-used i.e., a single package could contain enough coloring composition for several applications over time.

Thus, according to a further aspect of the present invention, there is provided a method for bleaching and/or colouring hair wherein a hair colouring and/or bleaching composition is applied directly to the hair and wherein the hair colouring and/or bleaching composition comprises:

(a) an oxidising agent;

(b) an oxidative hair coloring agent; and

(c) an alkoxylated benzoic acid.

(d)

The hair colouring and/or bleaching compositions herein are applied to the hair for periods of from 1 minute to 60 minutes depending upon the degree of coloring required. A preferred time is between 5 minutes and 30 minutes. The hair colouring and/or bleaching compositions according to the present invention can be applied to both wet and dry hair.

According to a still further and preferred aspect of the present invention, there is provided a method for bleaching and/or colouring hair comprising the steps of applying to the hair a first component which is an oxidising composition comprising an peroxygen bleach oxidising agent and an alkoxylated benzoic acid followed by applying to the hair a second component which is a dye composition comprising an oxidative hair coloring agent. In addition the present invention provides a method for bleaching and/or colouring the hair comprising the steps of applying to the hair a first component which is a dye composition comprising an oxidative hair coloring agent followed by applying to the hair a second component which is an oxidising composition comprising a peroxygen bleach oxidising agent and an alkoxylated benzoic acid.

Kits

According to the present invention there is also provided a hair colouring and/or bleaching kit wherein the kit comprises two or more separate components. In preferred embodiments the kit components are admixed to form a hair colouring and/or bleaching composition prior to application to the hair. In alternative embodiments, the individual components can be applied separately to the hair in a sequential manner.

A preferred kit herein comprises at least two components, a first component which is a dye composition containing an oxidative hair coloring agent and a second component which is an oxidising composition comprising a peroxygen bleach oxidising agent and an alkoxylated benzoic acid. The components can either be mixed by the user immediately prior to application to the hair or can be applied separately. Preferably the components are mixed by the user immediately prior to application to the hair. The components can also be mixed and the resulting composition can be stored for future use or part of the resulting composition can be used and the rest of the composition stored for future use. In one embodiment of the present invention the oxidising composition comprises a stabilised aqueous solution of an inorganic peroxygen oxidising agent, most generally hydrogen peroxide in an amount such that the final concentration of the hair colouring and/or bleaching composition for use on the hair is from about 0.01% to about 10%, preferably from about 0.01% to about 6%, more preferably from about 2% to about 5% by weight and additional agents as herein before described.

Examples of such kits are as follows:

I. A hair coloring kit is assembled comprising a single package including therein: (1) a 60 ml bottle of hydrogen peroxide (6% by weight of H₂O₂), an alkoxylated benzoic acid, and optionally buffering agents and/or stabilisers; and (2) a 60 ml bottle containing one or more oxidative hair coloring agents, and, optionally, additional agents such as surfactants, stabilisers, buffering agents, antioxidants, thickeners etc. The component containing the oxidative hair coloring agents can either be admixed with the hydrogen peroxide solution to form the dyeing system of the present invention and the resulting solution can be either applied to the hair to colour and/or bleach it or stored for future use, or the separately packaged stable components can be stored and mixed when required.

II. A hair coloring kit as described in I above wherein the hydrogen peroxide containing component is applied to the hair prior to application of the oxidative hair coloring agents, and additional materials to the hair.

III. A hair coloring kit as described in I above wherein the hydrogen peroxide containing component is applied to the hair after application of the oxidative hair coloring agents, and additional materials to the hair.

IV Further examples of kit components for the hair colouring and/or bleaching compositions according to the present invention include separately packaged oxidising composition and a separately packaged oxidative hair coloring agent component comprising hair coloring agent wherein either one or both components are present in particulate form.

What is claimed is:

1. A hair coloring and/or bleaching composition comprising a peroxygen bleaching agent, an alkoxylated benzoic acid and an oxidative hair coloring agent.

2. A hair colouring and/or bleaching composition according to claim 1 wherein said peroxygen bleaching agent is selected from inorganic peroxygen oxidising agents such as hydrogen peroxide, and preformed organic peroxyacids, and mixtures thereof.

3. A hair colouring and/or bleaching composition according to claim 1 or 2 wherein said alkoxylated benzoic acid or salt thereof has the general formula:

4. 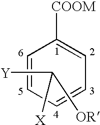
wherein the substituents of the benzene ring X and Y are independently selected from —H or —OR′ wherein R′ is independently selected from C₁ to C₂₀ linear or branched alkyl chains, and M is hydrogen, a cationic moiety or a cation.

5. A hair colouring and/or bleaching composition according to claim 3 wherein in the general formula the substituents of the benzene ring Y and X are —OR′, R′ is independently selected from C₁ to C₂₀ linear or branched alkyl chains, and M is hydrogen, a cationic moiety or a cation.

6. A hair colouring and/or bleaching composition according to claim 3 wherein in the general formula the substituents of the benzene ring X and Y are OR′, R′ is CH₃, and M is hydrogen, a cationic moiety or a cation.

7. A hair colouring and/or bleaching composition according to any of claims 1 to 5 wherein said alkoxylated benzoic acid or a salt thereof is 3,4,5-trimethoxy benzoic acid or a salt thereof.

8. A hair colouring and/or bleaching composition according to any of claims 1 to 6 wherein said composition has a pH in the range of from about 1 to about 12, preferably from about 1 to about 6, more preferably from about 1.5 to about 5.8, even more preferably from about 1.8 to about 5.5, especially from about 2 to about 5, and most preferably from about 3.5 to about 4.5.

9. A hair colouring and/or bleaching composition according to any of claims 1 to 7 wherein the peroxygen bleaching agent is present at a level of from about 0.01% to less than about 10%, preferably from about 0.01% to about 6%, more preferably from about 2% to about 5% by weight of the hair colouring and/or bleaching composition.

10. A hair colouring and/or bleaching composition according to any of claims 1 to 8 wherein the peroxygen bleaching agent is hydrogen peroxide.

11. A hair colouring and/or bleaching composition according to any of claims 1 to 9 wherein the oxidative hair coloring agent is present at a level of from about 0.001% to about 5%, preferably from about 0.01% to about 4%, more preferably from about 0.1% to about 3%, most preferably from about 0.1% to about 1% by weight of the hair colouring and/or bleaching composition.

12. A hair colouring and/or bleaching composition according to any of claims 1 to 10 additionally comprising a heavy metal ion sequestrant selected from sodium stannate, etidronic acid, disodium pyrophosphate, EDTA and pentasodium pentatate, and mixtures thereof.

13. Process of preparing the hair colouring and/or bleaching composition of any of claims 1 to 11 comprising the steps of preparing an oxidising composition and a dye composition, wherein the oxidising composition comprises a peroxygen bleaching agent and an alkoxylated benzoic acid and wherein the dye component comprises an oxidative hair colouring agent and then admixing the oxidising composition and the dye composition.

14. Use of a hair colouring and/or bleaching composition according to any of claims 1 to 11 for colouring or bleaching human or animal hair.

15. A method for colouring and/or bleaching human or animal hair comprising the steps of applying to the hair a colouring and/or bleaching composition according to any of claims 1 to 11, leaving said composition on the hair for 1 to 60 minutes, and then rinsing said composition from the hair.

16. A kit for colouring or bleaching the hair wherein the kit comprises an individually packaged oxidising composition and an individually packaged dye composition, wherein the oxidising composition comprises a peroxygen bleaching agent and an alkoxylated benzoic acid and wherein the dye composition comprises an oxidative hair colouring agent.

17. Use of a kit according to claim 15 for the bleaching and/or colouring of hair wherein the oxidising composition and the dye composition are admixed prior to application to the hair.
